(19) United States
(12) Patent Application Publication
(10) Pub. No.: US 2021/0052874 A1
(43) Pub. Date: Feb. 25, 2021

(54) ULTRASOUND DEVICE AND THERAPEUTIC METHODS

(71) Applicant: DREXEL UNIVERSITY, Philadelphia, PA (US)

(72) Inventors: Peter A. Lewin, Wyndmoor, PA (US); Elizabeth S. S. Papazoglou, Yardley, PA (US); Youhan Sunny, Philadelphia, PA (US); Chris R. Bawiec, Philadelphia, PA (US); Leonid Zubkov, Feasterville, PA (US); An Nguyen, Cherry Hill, NJ (US); Joshua Samuels, Philadelphia, PA (US)

(73) Assignee: DREXEL UNIVERSITY, Philadelphia, PA (US)

(21) Appl. No.: 17/072,435

(22) Filed: Oct. 16, 2020

Related U.S. Application Data
(63) Continuation of application No. 14/241,709, filed on Feb. 27, 2014, now abandoned.
(60) Provisional application No. 61/530,619, filed on Sep. 2, 2011.

(51) Int. Cl. A61M 37/00; A61N 7/00

(52) U.S. Cl. CPC A61M 37/0092; A61N 2007/0026; A61N 2007/0017; A61N 7/00

(57) ABSTRACT

The present invention is directed to an ultrasound device for use in implementing therapeutic treatments and transdermal analyte delivery. The device includes a piezoelectric transducer that efficiently and safely converts electrical energy to ultrasonic waves, and has a unique structure including a piezoelectric element positioned between two opposing, flexible concave covers. The device may be used for various therapeutic purposes including wound healing, tissue stimulation and transdermal analyte delivery. The invention is further directed to a novel analyte delivery system including the ultrasound device and an encapsulated analyte.

### STATEMENT OF GOVERNMENT INTEREST

This invention was made with Government support under Contract No. R01 EB009670-02 awarded by the National Institutes of Health. The Government has certain rights to this invention.

### BACKGROUND OF THE INVENTION

### 1. Field of the Invention

The present invention pertains to an ultrasound device and ultrasound mediated therapeutic treatment methods.

### 2. Brief Description of the Prior Art

Conventional sonicators are designed to generate acoustic energy at intensities well in excess of FDA approved guidelines of 100 mW/cm² for prolonged exposure and hence are unsafe and unable to efficiently generate ultrasound waves at low intensities and low pressure amplitudes. Furthermore, in order to generate prolonged high intensity acoustic output, these devices require large, heavy electronics to deliver large excitation voltages. Consequently, conventional sonicators are generally large, heavy, stationary structures that are not intended to be portable and do not provide a reasonable option for home treatment. While some ultrasound devices claim to be portable, at best they are bulky, unwieldy, rigid devices, easily weighing in excess of 40 lbs that and are not configured to be truly wearable or portable devices, such as a Band-Aid® like bandage or patch.

Conventional sonicators also do not enable control and adjustment of operational parameters, such as the duration of ultrasound administration, ultrasound frequency, ultrasound intensity, ultrasound pressure amplitude, transdermal delivery rate, and applied excitation voltage, and therefore do not allow for customized, individualized therapeutic treatment. Furthermore, these devices can also induce inertial cavitation resulting in potential tissue damage due to implosion of air voids near a cell wall or generation of free radicals. When used to mediate transdermal drug delivery, inertial cavitation can also cause the rupture of encapsulated drug molecules prior to passing through the epidermis. Consequently, conventional sonicators do not enable safe and intact drug delivery of encapsulated drug molecules.

For example, WO 97/04832 discloses a method for enhancing transdermal drug transport using low frequency ultrasound. The method involves using a portable sonicator capable of generating an acoustic intensity within the range of 12.5 mW/cm² to 225 mW/cm² and capable of generating an ultrasound frequency as low as 20 kHz. This reference, however, does not teach a transducer having a structure and capability to efficiently produce a low intensity acoustic output and low acoustic pressure amplitude from a minimal applied excitation voltage. Nor does it teach a truly wearable and portable device, such as a Band-Aid® like bandage or patch. Although the reference suggests using the sonicator for ultrasound mediated transdermal delivery of encapsulated drugs, it also does not demonstrate intact transdermal delivery of encapsulated drugs.

U.S. Pat. No. 6,190,315 discloses a method for transdermal transport of encapsulated drug molecules. The method involves applying low frequency ultrasound to the skin prior to and/or during transdermal delivery. Although the patent teaches that its transducer is capable of being operated at a frequency of 20 kHz to 2.5 MHz and capable of producing an ultrasound intensity of from 0 to 20 W/cm², it fails to teach a transducer having a structure and capability to efficiently produce a low intensity acoustic output and low acoustic pressure amplitude from a minimal applied excitation voltage. Furthermore, the device requires a bench-top ultrasound generator or a bulky and unwieldy portable ultrasound generator; it therefore does not teach truly wearable and portable applications, such as a Band-Aid® like bandage or patch. Furthermore, although the reference suggests using ultrasound mediated transdermal delivery of encapsulated drugs, it does not demonstrate intact transdermal delivery of encapsulated drugs.

U.S. Pat. No. 6,322,532 discloses a sonophoresis apparatus for transdermal drug delivery. The apparatus includes a flexible metal disk that is continuously joined to a piezoelectric disk which operates in flexural mode to produce a cavitation effect. During operation, 30V to 300V is applied to the transducer assembly to generate an acoustic output. Although the patent discloses that 0.05 W/cm² to 5 W/cm² of power is required to operate the apparatus, it does not specify either the acoustic intensity or the acoustic pressure amplitude generated by the transducer assembly nor the amount of voltage necessary to achieve the acoustic intensity or pressure amplitude. Moreover, it does not teach intact transdermal delivery of encapsulated drugs.

U.S. Pat. No. 7,429,249 discloses a wearable kit that utilizes low intensity ultrasound to induce stable cavitation and microstreaming for use in facilitating treatment of bone fracture and wound healing. It further discloses that the ultrasonic waves transmitted by the kit may be about 10 kHz to about 10 MHz and have SATA intensities of from about 5 mW/cm² to 500 mW/cm². This patent, however, does not contemplate the use of encapsulated drug molecules to achieve intact ultrasound mediated drug delivery. Furthermore, although the kit is intended to be worn by a patient, it requires the use of an unwieldy harness to attach it to a patient and can be awkward to use. Moreover, the patent does not teach transducers having a structure and capability to efficiently produce a low intensity acoustic output and low acoustic pressure amplitude from a minimal applied excitation voltage.

In view of the aforementioned deficiencies, there is a need to develop a portable, truly wearable, lightweight, ultrasound device that efficiently and safely enables non-invasive generation of low intensity and low pressure amplitude ultrasound waves for various therapeutic purposes. Additionally, there is a need to develop an improved method for ultrasound mediated transdermal drug delivery which permits intact transdermal delivery of encapsulated drug molecules.

### SUMMARY OF THE INVENTION

In a first aspect, the invention is directed to an ultrasound device for therapeutic treatment including an ultrasound transducer and a driving means that supplies an excitation voltage to the ultrasound transducer. The transducer has a piezoelectric element, a first cover and a second cover, wherein each cover has a concave configuration and wherein the piezoelectric element is positioned within a cavity formed by the opposing first and second covers.

In a second aspect, the invention is directed to a drug delivery system including an ultrasound device that facilitates transdermal drug delivery and an encapsulated drug positioned proximate to an ultrasound transducer. The ultrasound device includes an ultrasound transducer operatively associated with a driving means. The ultrasound transducer has a piezoelectric element, a first cover and a second cover, wherein each cover has a concave configuration and wherein the piezoelectric element is positioned within a cavity formed by the opposing first and second covers.

In a third aspect, the invention is directed to various therapeutic methods of using ultrasonic waves to treat a patient. The methods involve producing ultrasonic waves having an acoustic pressure amplitude of about 30 kPa to about 55 kPa upon applying an excitation voltage of about 10V to about 30V to a piezoelectric element of an ultrasound device positioned proximate to an epidermal surface for ultrasound mediated therapy.

### BRIEF DESCRIPTION OF THE DRAWINGS

FIG. 1(a) is a perspective view of an exemplary embodiment of one embodiment of the ultrasound device of the present invention.

FIG. 1(b) is a close-up of the ultrasound applicator array of FIG. 1(a).

FIG. 2(a) shows one embodiment of a modular ultrasound applicator.

FIG. 2(b) shows another embodiment of a modular ultrasound applicator configured as a parallelogram.

FIG. 3 is a perspective view of a transducer of the ultrasound applicator array.

FIG. 4(a) is a schematic diagram showing a cross-sectional view of a transducer at an initial state of rest (Stage 0) and subsequently contracted (Stage 1) to achieve a maximum vertical displacement and minimum radial extension corresponding to a positive cycle (peak) of excitation.

FIG. 4(b) is a schematic diagram showing a cross-sectional view of a transducer at an initial state of rest (Stage 0) and subsequently expanded (Stage −1) to achieve a maximum radial extension and minimum vertical displacement corresponding to a negative cycle (peak) of excitation.

FIG. 4(c) is a two dimensional symmetric model of a portion of the transducer operating at its fundamental mode of vibration showing a maximum displacement amplitude at the center.

FIG. 5(a) is a block diagram of an exemplary driving means.

FIG. 5(b) is a schematic diagram of another driving means operatively associated with an ultrasound applicator.

FIG. 6 is a perspective view of another embodiment of the ultrasound device having a rigid housing and flexible ultrasound applicator.

FIG. 7 is a schematic diagram of a drug delivery system.

FIG. 8(a) is a schematic diagram illustrating air voids within the stratum corneum.

FIG. 8(b) is a schematic diagram illustrating the formation of passageways from the air voids of FIG. 8(a) in the stratum corneum as a result of stable cavitation.

FIG. 9 is a graph showing acoustic pressure amplitude and acoustic intensity as a function of an applied excitation voltage comparing the ultrasound device of the present invention to conventional ultrasound devices described in literature.

FIG. 10 is a 3D representation of a 2D intensity field distribution (Iₛₚₜₚ) for a transducer in accordance with the invention.

FIG. 11 shows a 16.8 kHz pressure time waveform measured with a piezoelectric hollow cylinder hydrophone.

FIG. 12 shows an experimental setup for an in vitro study using an ultrasound device to deliver liposome encapsulated carboxyfluorescein (CF).

FIG. 13 is a graph of the concentration as a function of time comparing ultrasound assisted delivery of liposome encapsulated CF to unassisted delivery of unencapsulated CF (control).

FIG. 14 is a graph of the concentration as a function of time comparing ultrasound assisted delivery of liposome encapsulated CF to unassisted delivery of encapsulated CF as a function of time (control).

FIG. 15 shows an experimental setup using an ultrasound device to test the leakage of CF from liposome encapsulated CF.

FIG. 16 is a graph of 1,2-dioleoyl-sn-glycerol-3-phosphocholine (DOPC) percentage release as a function of ultrasound exposure time.

FIG. 17 is a graph of 1,2-dipalmitoyl-sn-glycerol-3-phosphocholine (DPPC) percentage release as a function of ultrasound exposure time.

### DETAILED DESCRIPTION OF THE PREFERRED EMBODIMENTS

For illustrative purposes, the principles of the present invention are described by referencing various exemplary embodiments. Although certain embodiments of the invention are specifically described herein, one of ordinary skill in the art will readily recognize that the same principles are equally applicable to, and can be employed in other systems and methods. Before explaining the disclosed embodiments of the present invention in detail, it is to be understood that the invention is not limited in its application to the details of any particular embodiment shown. Additionally, the terminology used herein is for the purpose of description and not of limitation. Furthermore, although certain methods are described with reference to steps that are presented herein in a certain order, in many instances, these steps may be performed in any order as may be appreciated by one skilled in the art; the novel method is therefore not limited to the particular arrangement of steps disclosed herein.

It must be noted that as used herein and in the appended claims, the singular forms “a”, “an”, and “the” include plural references unless the context clearly dictates otherwise. Furthermore, the terms “a” (or “an”), “one or more” and “at least one” can be used interchangeably herein. The terms “comprising”, “including”, “having” and “constructed from” can also be used interchangeably.

For purposes of the present invention, the term “stable cavitation” as used herein refers to air voids that have a tendency to increase in size and vibrate without imploding. The air voids vibrate when exposed a pressure field but do not implode. In stable cavitation a collection of air voids tend to operate in a relatively stable manner as long as a pressure field capable of producing rectified diffusion exits.

As used herein, the term “inertial cavitation” refers to the oscillation and violent collapse of air voids induced by an applied pressure field, usually at the air voids' resonance frequency. When the air voids implode near a cell wall, they exert a concentrated, high pressure force against the cell wall, which can destroy tissue and denature proteins in the cell. In addition to causing tissue damage, inertial cavitation may also generate free radicals.

As used herein, the terms “intact delivery,” “intact transdermal drug delivery,” or “delivery of intact encapsulated drugs” refers to the delivery of an analyte to a subcutaneous location, wherein the analyte has substantially the same ratio of chemical components and substantially the same concentration as prior to being administered by ultrasound mediated transdermal delivery. Intact drug delivery may be achieved using specific ultrasound frequency and acoustic field parameters such as for instance pressure amplitude or intensity and encapsulated drug s that substantially prevent analyte leakage during transdermal delivery thereby enabling analyte delivery in a predetermined concentration. Preferably, intact drug delivery limits analyte leakage from the vesicle to a maximum of about 10% or less, more preferably, about 6% or less, and most preferably, by about 4% or less.

The present invention is directed to a portable, truly wearable, lightweight ultrasound device that produces ultrasound waves for customized therapeutic treatment. Also, the invention is directed to methods for using the ultrasound device to treat wounds, stimulate tissue and enable substantially intact transdermal drug delivery of encapsulated drugs. Predicated on the importance of administering low intensity, low pressure amplitude, low frequency ultrasound waves generated from a minimal excitation voltage, the invention enables safe, effective and energy efficient ultrasound mediated therapy, and particularly, intact transdermal drug delivery. The invention also enables customization of ultrasound treatment and provides an ultrasound device that is easily portable, comfortably worn and easy to use, enabling effective therapeutic treatment and improved patient compliance.

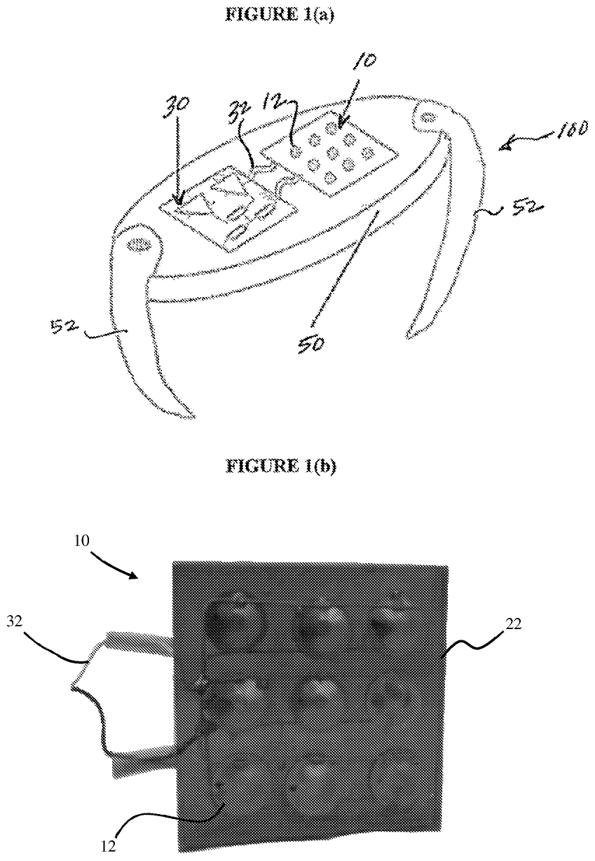

FIG. 1(a) shows an exemplary ultrasound device 100 including an ultrasound applicator 10 and an operatively associated driving means 30 positioned within or mounted to housing 50. Ultrasound applicator 10 has one or more transducers 12 adapted to efficiently generate a sufficient amount of acoustic energy to enable therapeutic treatment from a relatively low excitation voltage supplied by driving means 30. Transducers 12, shown in FIG. 3, each include a piezoelectric element 14 that is attached to and partially positioned within a cavity 20 defined by an assembly of first and second covers 16, 18. When excited, piezoelectric element 14 resonates in a flexural mode, and covers 16, 18 correspondingly vibrate. The flexural resonance of transducer 12 generates ultrasound waves for use in therapeutic treatment. The novel structure of transducers 12 amplifies the vertical and radial displacement amplitude of covers 16, 18 at their acoustic resonance, thereby enabling energy efficient generation of the ultrasound waves from an excitation voltage of about 10V to about 30V. In an exemplary embodiment, ultrasound applicator 10 is capable of generating a low intensity acoustic energy of about 0.1 mW/cm² to about 100 mW/cm², preferably, about 60 mW/cm² to about 100 mW/cm² and a low pressure amplitude of about 5 kPa to about 100 kPa, preferably, about 30 kPa to about 55 kPa. For applied voltages of at least 10V, ultrasound device 100 produces an acoustic intensity of at least 30 mW/cm² and a pressure of at least about 30 kPa.

Ultrasound device 100 preferably also enables a user or healthcare provider to control and adjust one or more parameters of driving means 30 in order to customize the therapeutic treatment. In an exemplary embodiment, ultrasound device 100 is configured as a flexible, lightweight, compact, portable, energy efficient device that can be comfortably worn by an individual and enables customized therapeutic treatment.

FIG. 1(b) shows an exemplary ultrasound applicator 10 of the present invention. As illustrated, ultrasound applicator 10 preferably includes a plurality of transducers 12 arranged in an array and operatively associated with driving means 30. In an exemplary embodiment, ultrasound applicator 10 may include about 1 to about 36 transducers 12, preferably about 4 to about 25 transducers, more preferably, about 4 to about 16 transducers, and most preferably about 9 to about 16 transducers forming an array. Preferably transducers 12 may be arranged in a series of rows and columns, such as a 2×2, 3×3 or 4×4 array, in a staggered pattern, in a radial pattern, in a series of concentric circles, or any arrangement that would facilitate therapeutic treatment. Preferably, the surface area of ultrasound device 100 occupied by one or more transducers 12 can be adapted to comply with and conform to the clinical application. Preferably, the surface area of ultrasound device 100 occupied by transducers 12, preferably ultrasound applicator 10, is about 200 mm² to about 3600 mm², preferably, about 200 mm² to about 1000 mm², more preferably, about 200 mm² to about 6400 mm², and most preferably about 1600 mm² to about 3600 mm².

In one embodiment, transducers 12 can be directly mounted to a surface of housing 50. For example, each transducer 12 may be potted within an opening or recess of housing 50 with an appropriate hardness controlled epoxy mixture in order to electrically insulate transducers 12, to electrically isolate covers 16, 18 and to encapsulate transducer 12 in a waterproof and structurally stable enclosure.

Optionally, as shown in FIG. 1(b), ultrasound applicator 10 may further include a frame 22 for mounting transducers 12 to housing 50. Frame 22 can be a flexible membrane that is preferably constructed from a non-conducting flexible material, such as cloth or silicone. The flexibility of frame 22 enables ultrasound applicator 10 to resiliently conform to the contours of an administration site. Alternatively, frame 22 may be rigid member preferably constructed from a non-conducting rigid material, such as a hard plastic material. Ultrasound applicator 10, including frame 22 and transducers 12, may be mounted within one or more cavities of housing 50. In an exemplary embodiment, ultrasound applicator 10 can be potted in a recess of housing 50 with an appropriate hardness controlled epoxy mixture to electrically insulate ultrasound applicator 10 form its surroundings. Preferably, ultrasound applicator 10 may be removably mounted to housing 50, thereby forming a modular device, wherein one or more ultrasound applicators 10 may be removably attached to housing 50 as desired. Ultrasound applicator 10 may be mounted to housing 50 using any conventional means including snaps, latches, quick connect fasteners, male and female fasteners, hook and loop fasteners, adhesives or any combinations thereof.

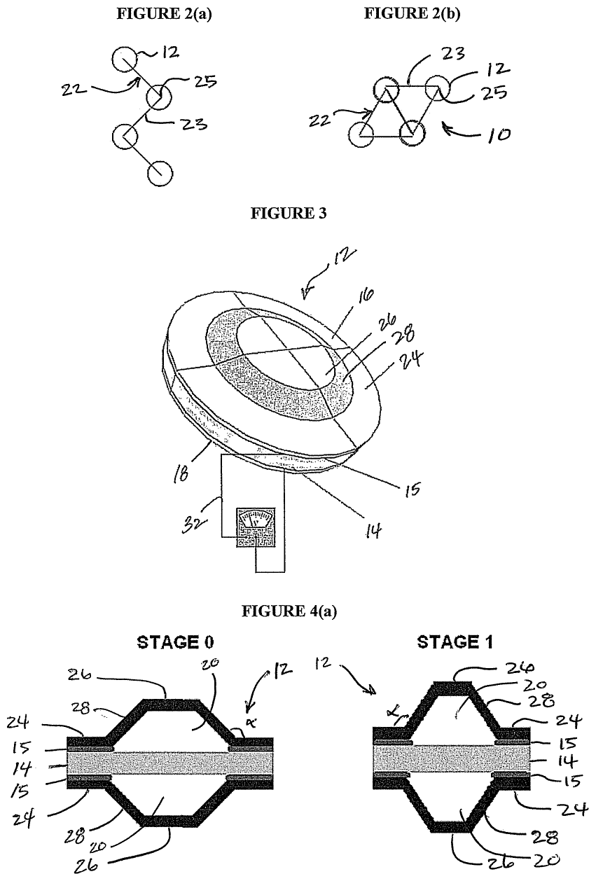

As shown in FIGS. 2(a)-2(b), ultrasound applicator 10 can have a modular configuration. In this embodiment, frame 22 is configured as an elongated flexible or rigid body comprising two or more segments 23, preferably a plurality of segments 23 are movable relative to one another. Adjoining segments 23 may be hingedly connected at a joint 25. One or more transducers 12 may be positioned along any portion of segments 23, preferably at a location proximate to ends of segments 23 forming joints 25. To maintain a desired configuration, segments 23 may include fasteners, such as snaps, latches or other locking mechanisms for fixing the position of two or more segments relative to one another. Alternatively, hinged joints 25 may have a sufficiently large coefficient of friction that segments 23 are caused to substantially maintain a fixed orientation. Upon applying a sufficient amount of force that overcomes the coefficient of friction of hinged joints 25, it is possible to adjust the orientation of segments 23.

The modular ultrasound applicator 10 may be separate from driving means 30. For example, modular ultrasound applicator 10 may be attached and/or worn by an individual, and driving means 30 may be removably connected to modular ultrasound applicator 10 to supply an excitation voltage.

FIG. 2(a) shows a modular ultrasound applicator 10 comprising three segments 23 including four separate transducers 12 two of which are positioned at opposite ends of each segment 23. The frame 23 is arranged in a first unfolded orientation. Frame 23 can be subsequently folded to form another configuration, such as a triangle.

FIG. 2(b) shows another ultrasound applicator 10 having five segments 23 arranged to form a parallelogram, wherein one of the segments 23 functions as a locking mechanism that attaches to and fixes two corners of the parallelogram to one another, thereby preventing the unfolding of frame 22. Additionally, two or more of these modular ultrasound applicators 10 may be coupled together to form different configurations.

In an exemplary embodiment, modular ultrasound applicator 10 includes a frame 22, configured as an elongated flexible wire body, and a plurality of transducers 12 mounted to the flexible, wire body of frame 22. In this embodiment, transducers 12 may be attached to and positioned on a patient in any desired configuration or arrangement. A thin film may be placed over ultrasound applicator 10 and wrapped around a portion of the patient to retain transducers 12 in a fixed position. Ultrasound applicator 10 may be then detachably connected to driving means 2 in order to initiate ultrasound therapy.

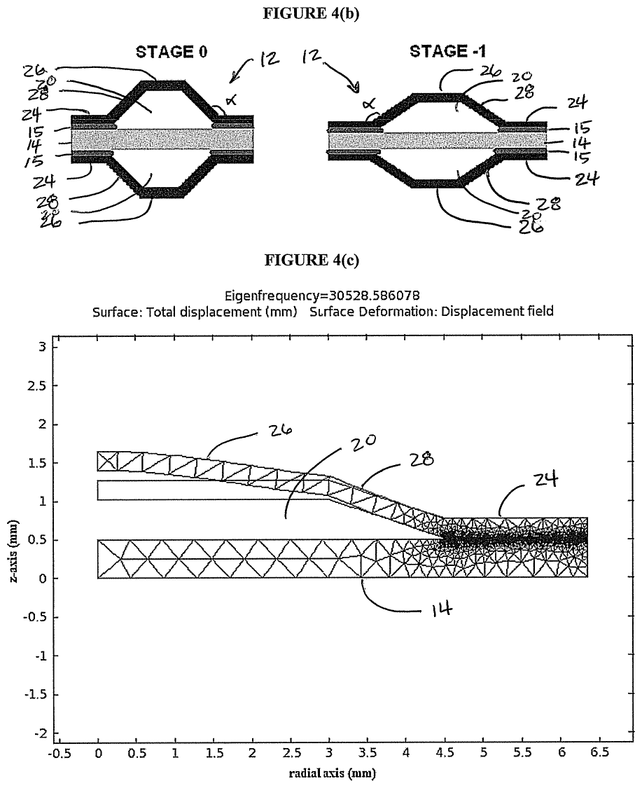

The structure of transducer 12 is best shown in FIGS. 3-4(c). Each transducer includes a piezoelectric element 14 that is positioned within a cavity 20 formed by first and second covers 16, 18. Piezoelectric element 14 is a piezoelectric plate, preferably configured as a thin disc that oscillates in a flexural mode when excited. In an exemplary embodiment, piezoelectric element 14 preferably has a thickness, an overall dimension and a piezoelectric coefficient sufficient to enable production of low intensity and low pressure amplitude ultrasound waves from a minimal amount of excitation voltage. Piezoelectric element 14 can constructed from any suitable piezoelectric material, including piezoelectric ceramics, such as lead zirconate titanate and hard lead zirconate titanate.

As shown in FIGS. 3 and 4(a)-4(b), two flexible covers 16, 18 are bonded to an upper and lower surface of piezoelectric element 14 by a conductive material. Covers 16, 18 serve to amplify the acoustic resonance of transducer 12. The flexibility and unique configuration of first and second covers 16, 18 permits covers 16, 18 to expand and contract in correspondence to the changes in the shape, size and spatial orientation of piezoelectric element 14 when excited. Specifically, the height and width of the covers 16, 18 changes as piezoelectric element 14 resonates, enabling transducer 12 to operate in a flextensional manner and achieve a maximum displacement amplitude using a relatively low excitation voltage.

First and second covers 16, 18 are preferably constructed from a highly flexible, electrically conductive material. Exemplary materials include metals, such as brass, copper, aluminum, stainless steel or titanium. In one embodiment, first cover 16 may be constructed from a different material or in a different thickness and may consequently have different flexibility properties than second cover 18.

Each cover 16, 18 has a concave configuration relative to the piezoelectric element 14 including a base 24 and a substantially dome shaped central region, defined by an apex 26 and one or more inclined side walls 28 connecting base 24 to apex 26. In the embodiment shown in FIG. 3, base 24 forms an outer perimeter of covers 16, 18, and is preferably configured as an annular plate having a substantially planar surface adapted to be bound to piezoelectric element 14. Piezoelectric element 14 has a diameter sufficient to achieve a desired ultrasound operational frequency. Spaced apart from base 24 is the apex 26 of the dome shaped region. In an exemplary embodiment, apex 26 may be a curved surface or planar surface. In the embodiment of FIG. 3, apex 26 is configured in a planar, circular configuration preferably having a diameter of about 0% to about 90%, more preferably, about 10% to about 50% and most preferably, about 20% to about 30% of the cover diameter. Inclined side wall 28 connecting base 24 to apex 26 allows covers 16, 18 to collapsible, wherein the height and width of covers 16, 18 expand and contract, as illustrated in FIGS. 4(a)-4(b).

Preferably, the length of inclined wall 28 between apex 26 and base 24 is about 0.1 mm to about 5 mm, more preferably, about 0.1 mm to about 2 mm and most preferably, about 0.2 mm to about 1 mm. In one embodiment, inclined wall 28 may move between a first position, wherein inclined wall 28 is substantially perpendicular to base 24, and a second position, wherein inclined wall 28 is substantially parallel to base 24. In another embodiment, the angle of inclination, a, between side wall 28 and base 24, as shown in FIG. 3, can change during flexural operation by about 5% to about 90° preferably, 5% to about 80°, more preferably, about 5% to about 70°. Additionally, the thickness of covers 16, 18, including inclined wall 28, is preferably about 0.01 mm to about 1 mm, more preferably, about 0.05 mm to about 0.5 mm, and most preferably, about 0.1 mm to about 0.3 mm.

During flexural operation, piezoelectric material 14 contracts to a smaller diameter causing the angle of inclination between base 24 and side wall 28 of the covers to decrease as piezoelectric element 14, as illustrated in FIG. 4(a), stage 1. Also, when piezoelectric element 14 expands in diameter, covers 16, 18 collapse towards one another, increasing the angle of inclination of side wall 28, as shown in FIG. 4(b), stage −1. In FIGS. 4(a)-4(b), stage 0 represents a non-excited state of rest occurring when no voltage is applied to piezoelectric element 14. Stage 1 represents a state of maximum vertical displacement over covers 16, 18 that occurs during positive cycles of excitation. Stage −1 represents a state of minimum vertical displacement of covers 16, 18 and maximum radial extension that occurs during a negative cycle of excitation. FIG. 4(c) illustrates the maximum displacement amplitude of apex 26 arising from the vibration of transducer 12, which is correlated to efficiency by which ultrasound applicator 10 is able to generate acoustic energy.

First and second concave covers 16, 18 directly face and oppose one another so as to form a cavity 20 for receiving piezoelectric element 14. Piezoelectric element 14 is positioned between first and second covers 16, 18, dividing cavity 20 into two separate compartments, as best illustrated in FIGS. 4(a)-4(b). The central region of piezoelectric element 14 is spaced apart from the apex 26 of first and second covers 16, 18, while a perimeter of piezoelectric element 14 is bonded to the base 24 using a conductive adhesive or other conductive bonding agent 15, such as a conductive epoxy. Preferably the outer perimeter of piezoelectric element 14 is coextensive with and bonded to the outer perimeter of covers 14, 16 so that that covers 16, 18 capture the movement of and efficiently amplify the displacement of piezoelectric element 14. At rest and during operation, the perimeter of covers 16, 18 are preferably coextensive with the perimeter of piezoelectric element 14, such that the diameter of piezoelectric element 14 is the same as that of covers 16, 18.

The structure and properties of transducer 12 enables the efficient generation of low acoustic intensity and low pressure amplitude ultrasound waves from a nominal applied excitation voltage. Preferably, transducer 12 enables the generation of FDA approved, acoustic intensities of about 0.1 mW/cm² to about 100 W/cm², more preferably, about 10 mW/cm² to about 100 mW/cm², more preferably, about 50 mW/cm² to about 100 mW/cm², and most preferably, about 60 mW/cm² to about 100 mW/cm² and low acoustic pressure amplitudes of about 5 kPa to about 100 kPa, preferably, about 5 kPa to about 80 kPa, more preferably about 30 kPa to about 55 kPa, and most preferably, about 40 kPa to about 55 kPa, at a low frequency of about 10 kHz to about 200 kHz, preferably, about 10 kHz to about 150 kHz, more preferably, 10 kHz to about 100 kHz and most preferably, about 20 kHz to about 100 kHz upon application of an initial excitation voltage of less than about 30V, preferably, less than about 20V and more preferably, less than about 10 V.

The energy efficiency of transducer 12 is dependent in part upon the material, geometry and dimensions of piezoelectric element 14, the material, structure and acoustic impedance of covers 16, 18, the volume and shape of cavity 20, how transducers 12 are mounted to housing 50 and the electrical matching network. Additionally, the generated ultrasound intensity, pressure amplitude and frequency is dependent upon the material, thickness and diameter of piezoelectric element 14, and the material, shape, cavity depth, outer diameter and apex diameter of first and second covers 16, 18.

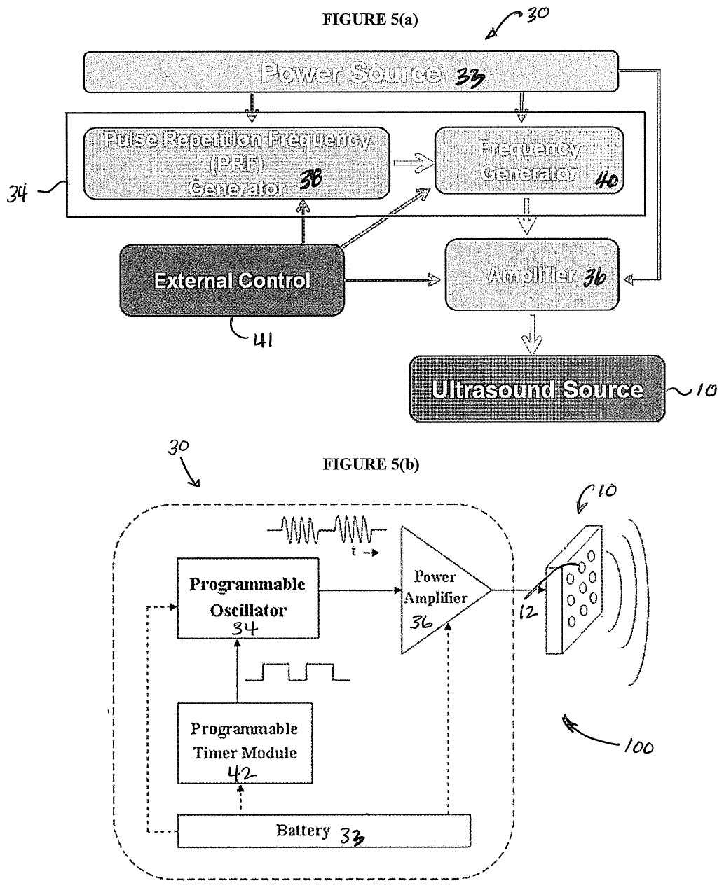

Ultrasound device 100 further includes a driving means 30, such as a electronic driving module, operatively associated with and connected to ultrasound applicator 10 through a matching network, such as inductors and/or resistors having various configurations and/or arrangements, and via electrical leads 32. Electrical leads 32 may be directly connected to piezoelectric element 14 or may be connected to first cover 16, second cover 18 or conductive bonding agent 15 to deliver an excitation voltage to transducers 12. In the embodiment shown in FIG. 5(a), driving means 30 includes a power source 33, an oscillator 34 and an amplifier 36. Power source 33 is preferably a small, portable rechargeable power source, such as a lithium battery, capable of supplying a sufficient amount of energy to operate ultrasound applicator 10 for at least about 6 hours, preferably at least about 12 hours, more preferably, at least about 24 hours and most preferably, at least about 48 hours. As a result of the highly efficient and low energy requirements of transducers 12, power source 33 may be a small battery of about 12V or less, preferably a battery of about 5V to about 11V, which substantially decreases the size and weight of ultrasound device 100, thereby enabling ultrasound device 100 to be configured as a portable and wearable apparatus capable of extended operation.

An oscillator 34 is connected to power source 33 and may include one or more components, such as a pulse repetition frequency generator 38 and frequency generator 40, for generating an electrical charge, as shown in FIG. 5(a). An amplifier 36 connected to the oscillator functions to adjust the current generated by the oscillator. As shown in FIG. 5(b), driving means 30 may optionally include a timer module 42 operatively associated with the oscillator that controls the duration that power is supplied to ultrasound applicator 10 and hence the duration of the therapeutic treatment. The oscillator 34, amplifier 36 and timer module 42 may be connected to a set of controls 41 that is either positioned on an exterior surface of housing 50 and/or remotely positioned relative to ultrasound device 100. Controls 41 enable a user or healthcare provider to set and adjust one or more of the ultrasound treatment duration, ultrasound frequency, acoustic intensity, acoustic pressure amplitude, transdermal delivery rate, and applied excitation voltage.

Driving means 30 and ultrasound applicator 10 are positioned within or otherwise mounted to housing 50 of ultrasound device 100. As shown in FIG. 1(a), housing 50 can be a flexible structure. Preferably, housing 50 is a flexible enclosure, such as a soft casing, pouch, patch or pad that is waterproof and made from cloth or a plastic material. The flexibility of housing 50 facilitates attachment to a patient and easily conforms to the contours of a treatment administration site. Preferably, housing 50 and ultrasound device 100 are configured as a thin and substantially planar bandage or patch, such as a BandAid® like implementation that may be fastened to a patient using any conventional means, such as adhesives.

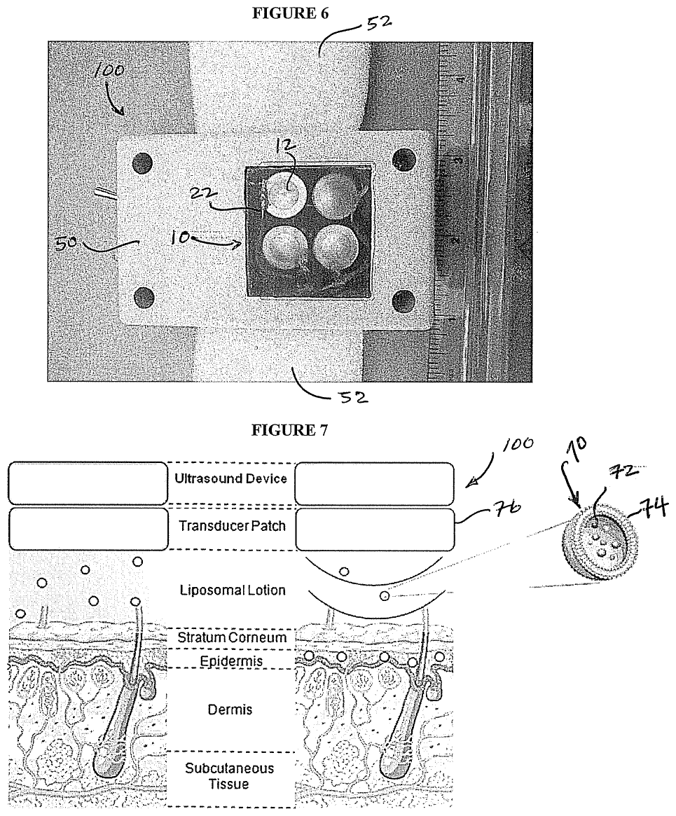

Alternatively, housing 50 can also be constructed as a rigid structure, as shown in FIG. 6. In this embodiment, housing 50 is a hard, waterproof plastic case but has a flexible frame 22 adapted to conform to a treatment administration site.

In one embodiment, flexible housing 50 includes one or more straps 52 to facilitate the removable attachment of ultrasound device 100 to a patient. To ensure that ultrasound device 100 is adequately secured to a patient, strap 52 may include a fastener 54 to removably attach strap 52 to a portion of ultrasound device 100 or to another strap 52 of ultrasound device 100. In the embodiments shown in FIGS. 1(a) and 6, two straps 52 positioned on opposite ends of ultrasound device 100 allow a patient to secure ultrasound device 100 about a patient's arms, legs or torso.

Optionally, ultrasound device 100 may further include a reservoir located within housing 50. Reservoir may contain a viscous medium, such as a gel, to increase acoustic coupling and facilitate ultrasound transmission. In one embodiment, reservoir may be located adjacent to ultrasound applicator 10, wherein the viscous medium is applied to a surface of transducers 12 upon manually squeezing reservoir or upon activating a pump.

In an exemplary embodiment, ultrasound device 100 is a light-weight, compact, easily portable device having a flexible housing 50. Preferably, ultrasound device 100 weighs about 200 grams or less, preferably, about 100 grams or less. Additionally, ultrasound applicator 100 preferably weighs about 60 grams or less, more preferably, about 40 grams or less and most preferably, about 15 grams or less. When using a modular ultrasound applicator, such as that of FIGS. 2(a)-2(b), the driving means 30 may be removably connected to ultrasound applicator 10 using an essentially weightless wire. Ultrasound device 100 preferably has a length of about 5 cm or less and a width of about 13 cm or less. Ultrasound device 100 also has a preferred thickness of about 5 cm or less. In one embodiment, ultrasound device 100 is configured as a substantially planar, flexible, lightweight adhesive bandage-like design.

Ultrasound device 100 of the present invention offers a number of advantages. By virtue of the novel structure of the piezoelectric transducers 10, ultrasound device 100 is capable of efficiently producing ultrasound waves having a low intensity of about 0.1 mW/cm² to about 100 mW/cm² and low acoustic pressure amplitude of about 5 kPa to about 100 kPa at a low frequency of about 10 kHz to about 200 kHz upon application of a minimum excitation voltage of about 30V or less. The structure and flexibility of transducer 12 substantially enhances the excitation voltage to ultrasound wave pressure amplitude efficiency, enabling the applicator to generate ultrasound waves having large amplitudes from relatively minimal voltage input. Operational safety is also enhanced by avoiding inertial cavitation, applying a low excitation voltage and generating ultrasound waves at low intensity and low pressure within FDA-approved guidelines that permits long term ultrasound exposure without undesirable side effects. Additionally the invention enables a healthcare provider or user to control and adjust the operating parameters of ultrasound device 100 to customize therapeutic treatment. Moreover, the lightweight, compact, flexible structure of ultrasound device 100, which preferably has a bandage or patch like design, is configured to be a portable, comfortable, discrete and truly wearable ultrasound delivery means. The invention therefore provides a safe, efficient, customized, non-invasive, pain-free, portable and wearable ultrasound mediated therapeutic treatment.

During operation, an electrical charge generated by driving means 2 is supplied to piezoelectric elements 14 of each transducer 12. Upon application of an initial excitation voltage of about 10V to about 30V, disk-shaped piezoelectric element 14 vibrates in a flexural mode, radially contracting, as illustrated in FIG. 4(a), and expanding, as illustrated in FIG. 4(b), to generate ultrasonic waves. First and second flexible covers 16, 18, which are bound to the outer perimeter of piezoelectric element 14, correspondingly change shapes as discussed above as piezoelectric element 14 oscillates, amplifying the acoustic resonance.

Upon application of an initial, low excitation voltage, ultrasound applicator 10 generates low intensity and low pressure amplitude ultrasound waves at a low frequency to allow for therapeutic treatment. To enable safe, FDA-approved ultrasound mediated therapeutic treatment, ultrasound applicator 10 preferably generates ultrasound waves having a pressure amplitude of about 55 kPa or less and a low acoustic intensity of about 100 mW/cm² or less at a frequency of about 200 kHz or less. In an exemplary embodiment, ultrasound applicator 10 preferably generates ultrasound waves having a low acoustic intensity of about 0.1 mW/cm² to about 100 mW/cm², more preferably, about 10 mW/cm² to about 100 mW/cm², more preferably, about 50 mW/cm² to about 100 mW/cm², and most preferably, about 60 mW/cm² to about 100 mW/cm² and a low pressure amplitude of about 5 kPa to about 100 kPa, preferably, about 5 kPa to about 80 kPa and more preferably, about 30 kPa to about 55 kPa and most preferably, about 40 kPa to about 55 kPa at a low frequency of about 10 kHz to about 200 kHz, preferably, about 10 kHz to about 150 kHz, more preferably, 10 kHz to about 100 kHz and most preferably, about 20 kHz to about 100 kHz upon applying initial excitation voltage of about 30V or less, preferably about 20V or less and more preferably, about 10 V or less. In an exemplary embodiment, ultrasound device 100 generates an acoustic intensity output to applied voltage ratio of at least about 0.37 mW/cm²/V or better, preferably, about 2.7 mW/cm² N or better, and most preferably, about 14.28 mW/cm²/V or better, more preferably, about 29 mW/cm² N or better and most preferably, about 67 mW/cm²/V or better. In an exemplary embodiment, ultrasound device 100 can have an excitation voltage to ultrasound wave amplitude efficiency of at least 20V:55 kPa/100 mWcm², preferably an excitation voltage to ultrasound wave amplitude efficiency of up to about 7V:55 kPa/100 mWcm². Ultrasound device 100 can also generates a pressure amplitude to applied voltage ratio of at least about 0.2 kPa/V or better, preferably, about 1.5 kPa/V or better, more preferably, about 7.8 kPa/V or better, more preferably, about 15 kPa/V or better, and most preferably, about 40 kPa/V. Ultrasound applicator 10 can also generate a continuous or pulsed ultrasound waves.

When ultrasound applicator 10 is positioned adjacent to an epidermal surface, the flexural vibration of transducers 12 induces acoustic tissue interactions that enable ultrasound mediated therapeutic treatment. Without wishing to be bound by theory, it is believed that the generated ultrasound waves: induce a substantial amount of stable cavitation within a patient's epidermal tissue and/or causes formation of sonopores through the epidermal tissue due to acoustic tissue interactions, such as applied radiation forces, acoustic streaming, shear stress or combinations thereof, that can induce different bioeffects on a cell membrane and cytoskeleton, sufficient to achieve a therapeutic effect and/or facilitate transdermal drug delivery. Preferably, transducers 12 may be operated at an acoustic intensity, pressure amplitude and frequency to generate a frequency, acoustic pressure amplitude and acoustic intensity sufficient to achieve and maintain substantial stable cavitation. Additionally, transducers 12 are preferably operated at an acoustic intensity, pressure amplitude and frequency that do not induce inertial cavitation, which can cause cellular tissue damage and inhibit intact drug delivery, as discussed in greater detail below.

In one embodiment, ultrasound device 100 may be used to facilitate wound healing. Ultrasound applicator 10 is positioned against an epidermal tissue of a patient proximate to the site of the wound, and a viscous medium may be spread over the transducers 12 and/or the epidermal tissue to facilitate ultrasound transmission. Ultrasound device 10 can then be used to achieve a therapeutic effect by applying low intensity and low pressure amplitude ultrasound waves to the site of the wound and/or by transdermally administering a drug to facilitate wound healing, as discussed in greater detail below. Using controls 41, a user or healthcare provider may specify and/or control one of more of the ultrasound treatment duration, ultrasound frequency, acoustic intensity, acoustic pressure amplitude, transdermal delivery rate, and applied excitation voltage to enable customized wound treatment. Additionally, a healthcare provider or user may monitor the user's response to the treatment and adjust one or more of these operational parameters as necessary based on the user's response.

Ultrasound device 100 operates in the same manner as that described above. Without wishing to be bound by theory, it is believed that the applied acoustic intensity, acoustic pressure amplitude and frequency induces a sufficient amount of substantial stable cavitation and/or formation of sonopores that facilitate wound healing and does not cause inertial cavitation. The ultrasound waves may be applied for a sufficient period of time to enable or facilitate wound healing. In an exemplary embodiment, wound healing is monitored after each treatment using a near infrared optic device that provides diagnostic information about the healing progress. Based on this information, the clinician can make a decision as to increasing or decreasing the number of, duration of or other operational parameter of treatment. Using this method, ultrasound device 100 may be used to treat various types of wounds, including chronic wounds, such as ulcers, particularly chronic ulcers symptomatic of diabetes.

In another embodiment, ultrasound device 100 may be used to stimulate tissue, specifically nerve tissue and organs. In this embodiment, ultrasound applicator 10 is positioned against an epidermal tissue of a patient proximate to the tissue to be stimulated. Where the tissue is an organ or nerve, ultrasound applicator 10 may be positioned over the patient's skin directly above the tissue to be treated. A viscous medium may be spread over the transducers 12 and/or epidermal tissue to facilitate ultrasound transmission. Ultrasound device 10 may then be used to achieve a therapeutic effect by applying low intensity and low pressure amplitude ultrasound waves to the tissue. Using controls 41, a user or heath care provider may specify and/or control one or more of an ultrasound treatment duration, ultrasound frequency, acoustic intensity, acoustic pressure amplitude, transdermal delivery rate, and applied excitation voltage to enable customized therapeutic treatment. Additionally, a user or healthcare provider may adjust one or more of these operational parameters during treatment based on the user's response to the ultrasound therapy.

Without wishing to be bound by theory, it is believed that the applied low intensity, low pressure amplitude and s and low frequency ultrasound waves induces a sufficient amount of stable cavitation and/or formation of sonopores that facilitates tissue stimulation and does not cause inertial cavitation. The ultrasound waves may be applied for a sufficient period of time to enable or facilitate tissue stimulation. In an exemplary embodiment, tissue stimulation is monitored after each treatment using a near infrared optic device that provides diagnostic information about the degree of tissue stimulation. Based on this information, the clinician can make a decision as to increasing or decreasing the number of, duration of or other operational parameter of treatment. Using this method, ultrasound device 100 may be used to treat any nerve, including static nerves and nerves that have been damaged by diabetic neuropathy or trauma, such as the nerves within a patient's hands, legs, feet and other extremities, as well as treat organs having impaired functionality.

As discussed above, the invention is also directed to a novel method and system for transdermal drug delivery. In this embodiment, the drug delivery system including ultrasound device 100 and vesicle encapsulated analyte 70.

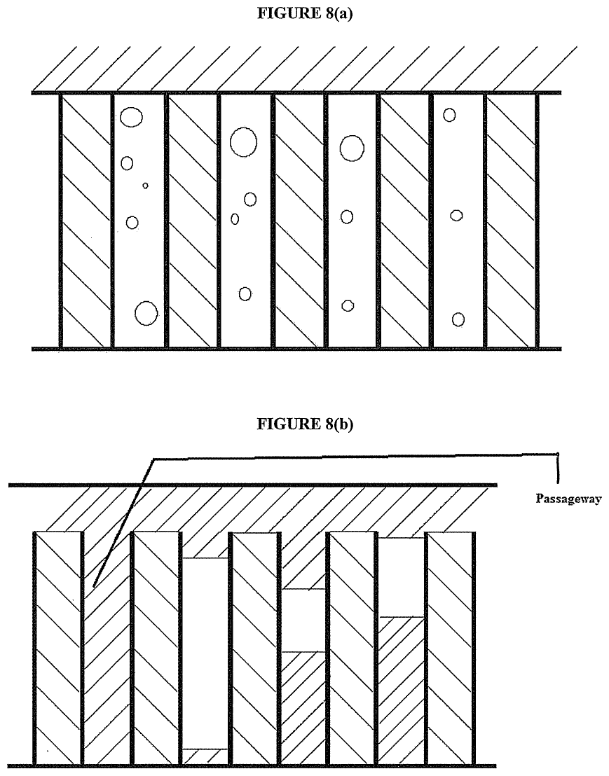

As best shown in FIG. 7, vesicle encapsulated analyte 70 includes one or more analytes 72 encapsulated within a vesicle 74. Exemplary analytes 72 may include pharmaceuticals, si-RNA, antibodies, proteins, hydrophobic molecules, hydrophilic molecules, arthritis drugs, such as methotrexate, cancer drugs, anti-inflammatory rheumatic drugs, pain medication, antibiotics, drugs that minimize the side-effects of chemotherapy and wound management drugs.

Vesicle 74 provides a protective shell that facilitates transdermal permeation, and may be used to control analyte release once the vesicle encapsulated drug 70 has reached a target site, such as a patient's blood stream. The nanoarchitecture of vesicle 74 is designed to ensure the safe delivery of analyte 72 to a target location in substantially the same concentration and molar ratio as is present prior to ultrasound mediated transdermal administration. Vesicle 74 may have a size and chemistry designed to achieve a membrane curvature, fluidity and bending rigidity that enables transdermal delivery without substantial leakage or rupture upon insonificiation. Preferably, vesicle 74 is constructed from lipids selected based on phase behavior, elastic modulus and membrane viscosity appropriate for the intended ultrasound mediated transdermal application and for the analyte to be delivered. Exemplary lipids used to construct vesicle 74 may include 1,2-dioleoyl-sn-glycerol-3-phosphocholine, 1,2-dipalmitoyl-sn-glycerol-3-phosphocholine, 1,2 dipalmitoleoyl-sn-glycero-3-phosphocholine and 1,2-dimyrstoyl-sn-glycero-3-phosphocholine or combinations thereof. Vesicle 74 may be configured as a liposome, polymeric nanoparticles, polymersomes, microparticles, microcapsules, microspheres or similar encapsulation vehicles. Preferably, vesicle 74 may be configured as a uni-lamellar structure having a diameter of about 1 um or smaller, more preferably, about 300 nm or smaller and most preferably, about 100 nm or smaller. In an exemplary embodiment, vesicles 72 are constructed in accordance with nanoparticle critical design parameters that substantially prevent or minimize leakage or rupturing when exposed to low intensity and low pressure amplitude ultrasound waves at low frequencies and when exposed to stable cavitation. Preferably, vesicles 72 are deigned to substantially resist leakage or rupture when exposed to inertial cavitation.

As illustrated in FIG. 7, vesicle encapsulated analyte 70 is optionally contained within a flexible and porous casing 76, such as a patch, pouch or sack that may be removably mounted to ultrasound applicator 10. Preferably, vesicle encapsulated analyte 70 is interspersed within a viscous medium that facilitates ultrasound administration, such as a gel. Casing 76 may include one or more openings for receiving one or more transducers 12. When casing 76 is mounted to ultrasound applicator 10, transducers 12 are immersed within and are in direct contact with the encapsulated drug dispersed viscous medium contained in casing 76. Casing 76 further includes a porous surface that permits permeation of vesicle encapsulated analyte 70 therethrough but preferably prevents permeation of the viscous medium.

During operation, casing 76 containing the encapsulated drug dispersed viscous medium is coupled to ultrasound applicator 10 and positioned against an epidermal tissue of a patient. Using controls 41, a user or heath care provider may then specify and/or control one or more of an ultrasound treatment duration, ultrasound frequency, acoustic intensity, acoustic pressure amplitude, transdermal delivery rate, and applied excitation voltage to enable customized drug delivery. Additionally, a user or healthcare provider may adjust one or more of these operational parameters during drug delivery.

Without wishing to be bound by theory, it is believed that the applied low intensity, low pressure amplitude and low frequency ultrasound waves of the device induces a sufficient amount of stable cavitation and/or formation of sonopores to facilitate intact delivery of vesicle encapsulated analyte 70. It is further believed that the applied ultrasound waves do not cause inertial cavitation. Without wishing to be bound by theory, it is believed that stable cavitation induces rectified diffusion and pushes the vesicle encapsulated analyte 70 through the stratum corneum of a patient's skin. FIG. 8(a) shows air voids in the stratum corneum that expand to form channels, illustrated in FIG. 8(b). Specifically, FIGS. 8(a)-8(b) shows how rectified diffusion may play an important role in sonophoresis by causing bubbles in the stratum corneum to grow and ultimately fuse, creating channels through which particles can more easily pass. The phenomenon of rectified diffusion and stable cavitation is further explained in Lavon, I., et. al., “Bubble growth within the skin by rectified diffusion might play a significant role in sonophoresis,” Journal of Controlled Release, 117, 2007, herein incorporated by reference in its entirety.

Another mechanism of action by which the ultrasound device 100 enables transdermal transport involves the formation of sonopores when ultrasound field interacts with a tissue bilayer, which induces stretching and the pulling apart of the tissue membrane. This disrupts the structure of the stratum corneum, thereby forming pores or causing rupturing of the membrane enabling passage of the vesicle encapsulated analyte 70. This phenomenon is further explained in Krasovitski, B. et al., “Intramembrane cavitation as a unifying mechanism for ultrasound-induced bioeffects,” PNAS, 2011, herein incorporated by reference in its entirety.

The present invention therefore ensures substantially intact delivery of vesicle encapsulated analyte 70 to a target site, such as the blood stream, in a predetermined and preferably uniform concentration. Vesicle 74 enables analytes 72 to be transdermally delivered substantially intact and in sustained, clinically critical concentrations. Additionally, by operating ultrasound device 100 at a low acoustic intensities and a low frequency range, without inducing inertial cavitation, the invention enables stratum corneum penetration and sustained, stable drug delivery to a target site.

### EXAMPLES

### Example 1

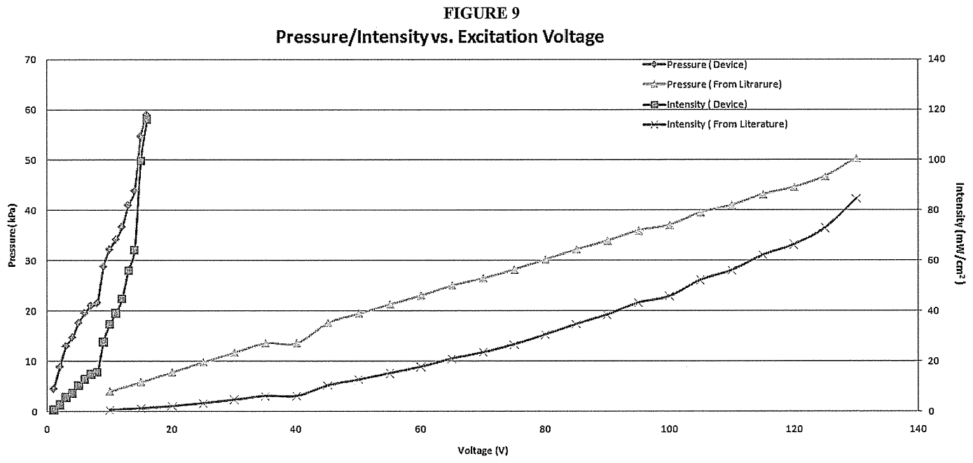

A study investigating the efficiency of the ultrasound device of the present invention to generate acoustic energy from a nominal applied voltage was performed. An ultrasound device 100 having an ultrasound applicator 10 including a 2×2 array of four piezoelectric transducers 12 was used. Voltages over a range of about 0V to 20V were applied to the piezoelectric elements 14 of the transducers 12. As shown in FIG. 9, the ultrasound applicator 10 produced low intensity acoustic output of about 60 mW/cm² to about 100 mW/cm² and low pressure ultrasound waves of about 30 kPa to about 50 kPa in response to the applied excitation voltages.

In comparison, FIG. 9 also shows that conventional sonicators discussed in the literature, which are designed and intended for high intensity acoustic output applications; these conventional devices are therefore inefficient at generating low intensity and low pressure ultrasound waves. FIG. 9 illustrates that these conventional sonicators typically require in excess of 120 V to generate ultrasound waves having an acoustic intensity of about 90 mW/cm² and a pressure amplitude of about 50 kPa.

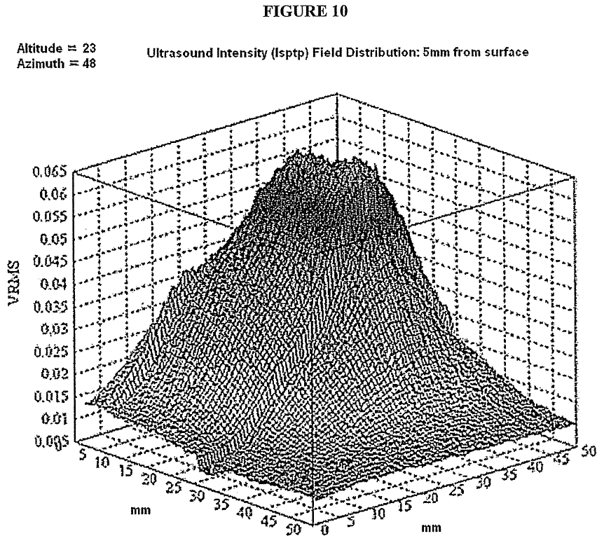

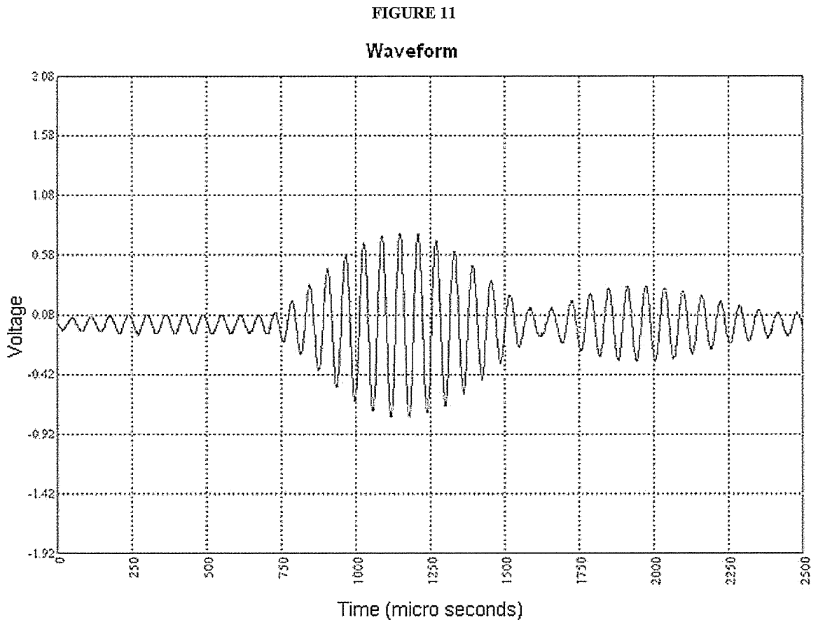

FIG. 10 shows a 3D model of a 2D intensity field distribution of the acoustic energy produced by the transducer 12 at a distance of about 5 mm away from the transmitting surface of the transducer 12. FIG. 11 shows a 16.8 kHz pressure time waveform corresponding to a peak-to-peak pressure amplitude of about 55 kPa measured at a clinically relevant axial distance of about 5 mm in response to an initial excitation voltage of about 16 mV at about a 60 dB attenuation.

### Example 2

An in vitro study was performed to evaluate the ability of the present invention to enable transdermal delivery of substantially intact encapsulated drugs. The study involved applying low intensity, low pressure amplitude ultrasound waves to mouse skin in order to mediate transdermal delivery of liposome encapsulated carboxyfluorescein (CF), a hydrophilic dye.

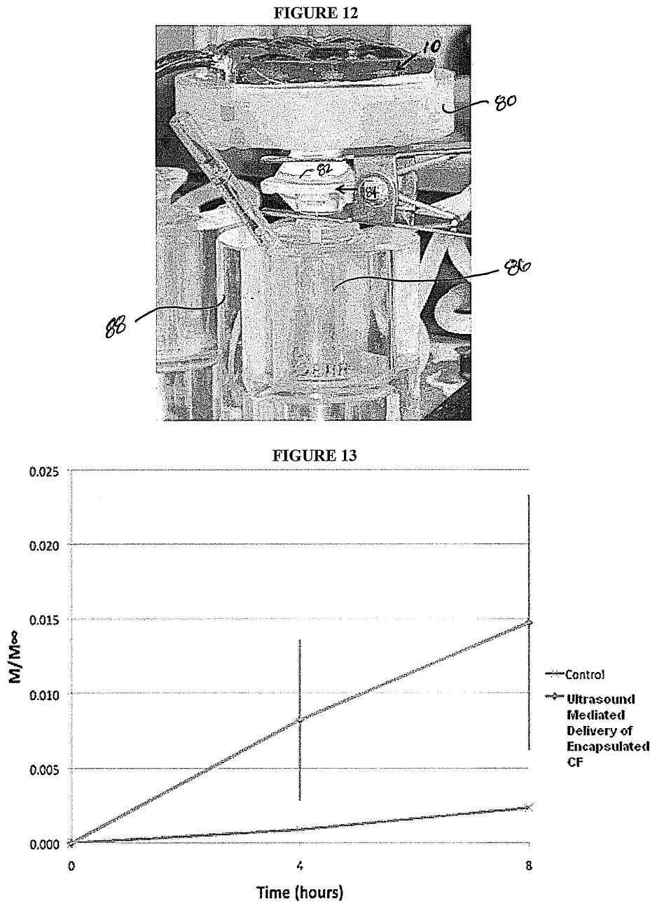

FIG. 12 shows the experimental setup used in the investigation. In this setup, an ultrasound applicator 10 having 4 transducers 12 arranged in a 2×2 array is supported by a custom fixture 80 and in direct communication with a Franz diffusion cell, including a donor compartment 82 filled with a liquid medium interspersed with liposome encapsulated CF and a sample mouse skin. Low frequency ultrasound waves (LFUS) having a low pressure amplitude of about 55 kPa, a low acoustic intensity of about 100 mW/cm² and a low frequency of about 17.9 kHz was generated by ultrasound applicator 10 and directed towards a mouse skin sample 84 positioned adjacent to donor compartment 82 for about 4 hours. The amount of liposome encapsulated CF that permeated mouse skin 84 and collected within a receptor compartment 86 surrounded by a water jacket 88 was examined and measured after about 4 hours of ultrasound application when the ultrasound source was turned off and again 8 hours after the start of the experiment.

The liposomes were made from 1,2-dioleoyl-sn-glycerol-3-phosphocholine (DOPC), and the liposome encapsulated CF was prepared using the dehydration-rehydration technique described in Kirby, C. and G. Gregoriadis, “Dehydration rehydration vesicles: a simple method for high yield drug entrapment in liposome,” Nature Biotechnology, 1984, 2(11): p. 9′79-984, herein incorporated by reference. In this experiment, encapsulation efficiency of the CF was improved (about 10% to about 30% measured by titration) by minimizing the amount of rehydration buffer used, adjusting pH before rehydration to fully dissolve CF by minimizing the amount of rehydration buffer used, and by adjusting the pH to about 10 and subsequently adjusting the pH to about 7.4 before rehydration. The original encapsulation efficiency of CF was about 0.67% in 100 nm liposomes, using about 5.2 mg CF in the rehydration buffer (about 2.56 mM).

The same experimental setup was also used to evaluate the transdermal penetration of unencapsulated CF that was not exposed to ultrasound and the transdermal penetration of encapsulated CF that was not exposed to ultrasound

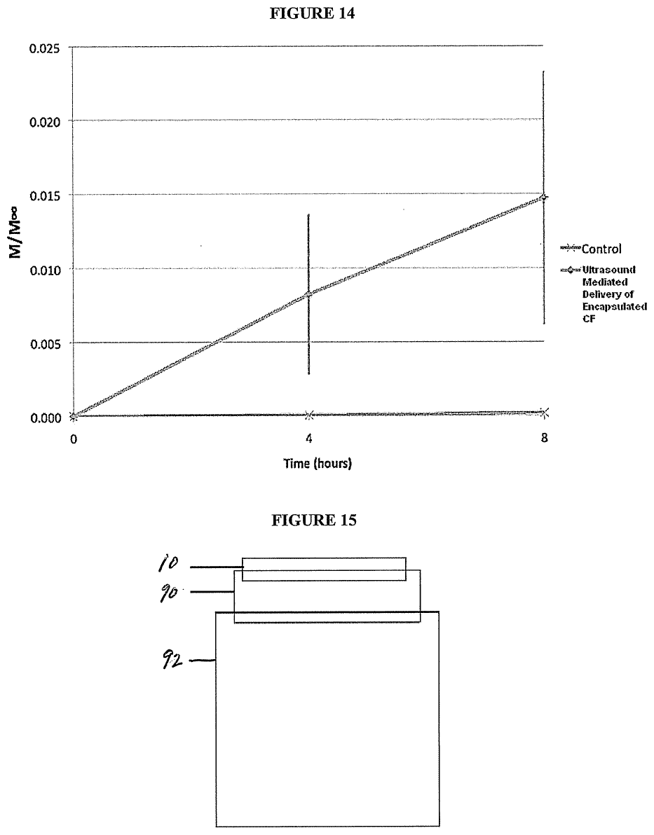

FIGS. 13 and 14 show that a substantial amount of liposome encapsulated CF successfully permeated the mouse skin when transdermal delivery was facilitated by the method of the present invention. Examination of the liposome encapsulated CF collected within receptor compartment 88 further confirmed that the liposomes of the encapsulated CF remained substantially intact after transdermal delivery.

FIGS. 13 and 14 also compare ultrasound mediated delivery of the liposome encapsulated CF to the permeation of unencapsulated CF and encapsulated CF delivered without the assistance of ultrasound. Notably, only a nominal amount of unencapsulated CF less than 0.005 M/M and no encapsulated CF was able to permeate the mouse skin after 8 hours when not assisted by ultrasound meditation. By comparison, 0.015 M/M of the ultrasound mediated liposome encapsulated CF was able to penetrate the mouse skin after 8 hours.

Table 1 shows the enhancement ratios, defined in equation 1 below, and delivery efficiency results from the experiment.

$$Enhancement Ratio = \frac{\% of CF delivered}{\% of CF delivered with free CF , no LFUS} \qquad Equation 1$$

Test A shows the results for unencapsulated CF without the application of ultrasound. Test B shows the results for unencapsulated CF upon exposure to 4 hours of low frequency ultrasound. Test C shows the results for liposome encapsulated CF without the application of ultrasound. Tests D1-D2 show the results for liposome encapsulated CF after exposure to 20 minutes of low frequency ultrasound stimulation. Tests E1-3 show the results for liposome encapsulated CF upon exposure to 4 hours of low frequency ultrasound stimulation.

TABLE 1
Enhancement of Carboxyfluorescein delivery using a 20 kHz
ultrasound transducer array

| | | Enhancement | Delivery |
|---|---|---|---|
| | | Ratio v. free CF, | Efficiency (% of |
| Test | Description | no LFUS | CF delivered) |
| A | Free CF, no LFUS | 0.00 | 0.03 |
| B | Free CF, 4 hr | 0.00 | 0.03 |
| | simultaneous LFUS | | |
| C | CF in liposomes, no LFUS | 10 | 0.3 |
| D1 | CF in liposomes, | 23 | 0.7 |
| | 20 min simultaneous LFUS | | |
| D2 | CF in liposomes, | 40 | 1.2 |
| | 20 min pretreatment LFUS | | |
| E1 | CF in liposomes, | 103 | 3.2 |
| | 4 hr simultaneous LFUS | | |
| E2 | CF in liposomes, | 57 | 1.7 |
| | 4 hr simultaneous LFUS | | |
| E3 | CF in liposomes, | 40 | 2.1 |
| | 4 hr simultaneous LFUS | | |

### Example 3

A similar experiment set-up as described in Example 2 was used to investigate the effect of liposome size and viscosity on transdermal delivery. Unlike the experimental set-up in Example 2, the set-up here did not require a custom fixture for the ultrasound source as no ultrasound source was involved in this Example. Specifically, unassisted diffusion of drug filled liposomes constructed from 1,2-dioleoyl-sn-glycerol-3-phosphocholine (DOPC) and 1,2-dipalmitoyl-sn-glycerol-3-phosphocholine (DPPC) through human skin samples were tested.

The liposomes were prepared using the dehydration-rehydration technique described in Kirby, C. and G. Gregoriadis, “Dehydration rehydration vesicles: a simple method for high yield drug entrapment in liposome,” Nature Biotechnology, 1984, 2(11): p. 979-984, herein incorporated by reference.

The results showed that by decreasing liposme size from 200 nm to 50 nm, there was a doubling of the delivery rate, irrespective of the type of liposome bilayer, e.g. DOPC v. DPPC, used. On the other hand, the type of liposome bilayer used effected delivery rate for a given vesicle size. In particular, DOPC bilayers exhibited twice the rate of delivery than DPPC bilayers at all vesicle sizes tested. This was attributed to the fact that DOPC bilayers are in the liquid disordered phase and therefore more fluid than the DPPC ones which are in the liquid ordered phase; clearly viscosity influences the passive diffusion rate.

### Example 4

Another study investigated the ability of specific liposomes to prevent leakage of an encapsulated analyte upon application of a low pressure amplitude ultrasound wave of about 55 kPa and an acoustic intensity of about 100 mW/cm² at a frequency of about 17.9 kHz. In this experiment, an ultrasound applicator 10 having 4 transducers 12 arranged in a 2×2 array was positioned adjacent to a PetriDish 90 containing liposome encapsulated carboxyfluorescein (CF), as shown in FIG. 15. The PetriDish 90 had an open upper end positioned adjacent to ultrasound applicator 10 and a closed lower end positioned within a water bath 92 filled with 32° C. water. The fluorescence levels of the CF were examined prior to and after ultrasound exposure. The results confirmed that the liposomes remained substantially intact after ultrasound exposure and did not exhibit a significant amount of leakage.

In this experiment, two liposomes were investigated. The liposomes were synthesized from two different lipid materials, namely 1,2-dioleoyl-sn-glycerol-3-phosphocholine (DOPC) and 1,2-dipalmitoyl-sn-glycerol-3-phosphocholine (DPPC). DOPC formed a fluid membrane at skin temperature and had a T. of about −20° C. DPPC formed a rigid membrane at skin temperature and had a T. of about 42° C.

The liposomes were prepared using the dehydration-rehydration technique described in Kirby, C. and G. Gregoriadis, “Dehydration rehydration vesicles: a simple method for high yield drug entrapment in liposome,” Nature Biotechnology, 1984, 2(11): p. 979-984, herein incorporated by reference.

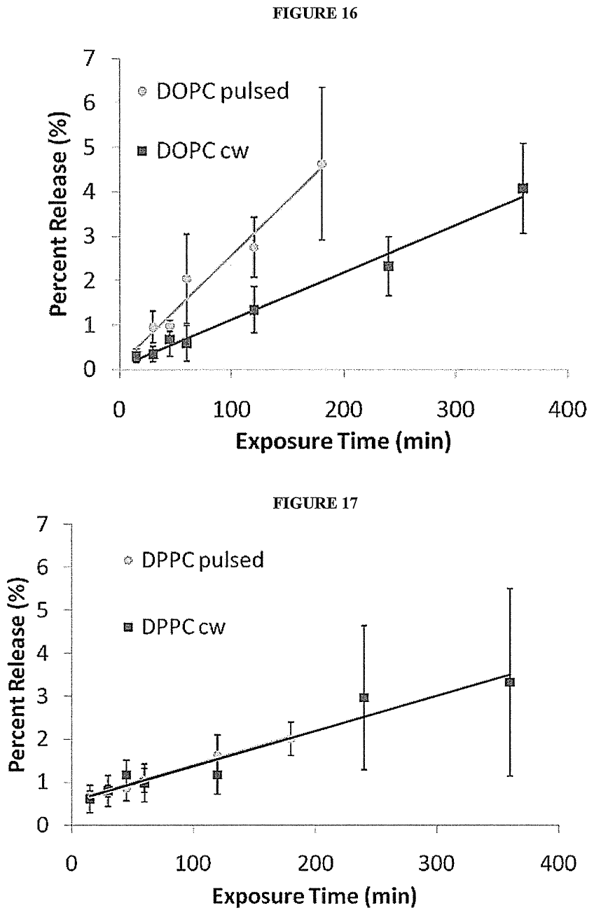

FIGS. 16-17 show the percent release rate of the DOPC and DPPC liposome encapsulated CF as a function of time. Leakage rates depended on temporal (continuous wave (CW) vs. pulsed) delivery fro DOPC liposomes and is unaffected by pulse rate for DPPC liposomes. Maximum leakage occurred after pulsed sonification of 3 hours (40% duty cycle), with a leakage rate of approximately 4% in DOPC liposomes. The results showed that the amount of CF released was negligible. Specifically, the DOPC liposomes experienced a maximum leakage rate of about 4.6% when exposed to pulsed ultrasonic waves and about 4% when exposed to non-pulsed ultrasonic waves at a low pressure amplitude of about 55 kPa and at a low intensity of about 100 mW/cm² a frequency of about 17.9 kHz.

DPPC liposomes had a maximum leakage rate of about 3.3% when exposed to either pulsed or non-pulsed ultrasonic waves having a low pressure amplitude of about 55 kPa and an intensity of about 100 mW/cm² at a frequency of about 17.9 kHz. These statistically insignificant, low leakage rates demonstrate that the applied acoustic energy does not cause significant leakage of CF from the liposome.

### Example 5

Twenty patients that had a venous ulcer for at least 6 weeks and larger than 1 cm² and were between the ages of 18 and 65 were treated. Consenting subjects were randomly assigned to one of 4 different ultrasound treatment groups: 15 minutes of sham, 15 minutes of 20 kHz ultrasound, 45 minutes of 20 kHz ultrasound, or 15 minutes of 100 kHz ultrasound. All active treatments had an intensity of 100 mW/cm² SPTP with a duty cycle of 50%. Five patients were enrolled in each group. Topical analgesic was applied to the wounds prior to each treatment. A non-invasive optical measurement (Diffuse Near Infrared Spectroscopy) was taken on the wound before and after ultrasound treatment to quantify oxy- and deoxy-hemoglobin concentrations. The ultrasound transducer of the invention was then applied to the wound with a small amount of sterile ultrasound gel as a coupling medium. The transducer was taped to the leg for the duration of the ultrasound treatment. In the case of the sham treatment, a non-functioning ultrasound device identical in form to the functioning ultrasound transducers was applied to the wound using the same procedure. A photograph was taken after each treatment to monitor changes in wound size. All treatments, measurements and photographs were taken prior to debridement by the physician.

1 out of the 5 wounds in the sham (non-treatment) group healed whereas 9 out of the 15 wounds sonicated healed in the 4 week time frame. The optimal group, which was 20 kHz treatment for 15 minutes, had 5 out of the 5 patients heal in 4 weeks, representing a 100% success rate. Every ultrasound treatment group had a higher healing rate than the control group.

The foregoing examples have been presented for the purpose of illustration and description and are not to be construed as limiting the scope of the invention in any way. The scope of the invention is to be determined from the claims appended hereto.

1-15. (canceled)

16. A therapeutic method to treat a patient having a wound with a device by ultrasound mediated therapy, wherein the method comprises the step of:
producing ultrasonic waves with the device operating at an excitation voltage of 20V or less, said ultrasonic waves having an acoustic pressure amplitude of 5 kPa to 100 kPa, an acoustic intensity of 0.1 mW/cm² to 100 mW/cm², and a frequency of 10 kHz to 200 kHz, wherein the device is positioned proximate to a site of the wound for a sufficient time to achieve a therapeutic wound healing effect by exposing the wound to the ultrasound waves,
the device comprising:
at least one ultrasound transducer, wherein the ultrasound transducer comprises:
a first flexible cover having a concave configuration;
a second flexible cover having a concave configuration opposing the first cover to form a cavity between said first and second covers, and
a piezoelectric element attached to and positioned between the first and second covers; and
an electronic driving module connected through an electrical matching network and operatively associated with at least one ultrasound transducer to supply an excitation voltage of 20V or less to the matching network connected to the piezoelectric element,
wherein the first and second covers are fabricated from a conductive material and a perimeter of the piezoelectric element is bonded to the first and second covers by an adhesive layer formed of a conductive material,
the electronic driving module comprises an oscillator, and
the ultrasound transducer is mounted to a flexible membrane that conforms to the surface of the site of administration.

17. The method of claim 16, further comprising the step of transdermally administering an encapsulated analyte using the ultrasound device to enhance transdermal delivery of said encapsulated analyte.

18. The system of claim 17, wherein the encapsulated analyte comprises a vesicle selected from the group consisting of: liposomes, polymeric nanoparticles, microparticles, microcapsules, microspheres and combinations thereof.

19. The method of claim 16, further comprising the step of adjusting one or more operating parameters of the ultrasound device selected from the group consisting of: duration of ultrasound administration, ultrasound frequency, ultrasound intensity, ultrasound pressure amplitude, transdermal delivery rate, and applied excitation voltage.

20. (canceled)

21. The method of claim 16, wherein the device is positioned with the membrane in contact with the site of the wound such that the membrane conforms to contours of the site of the wound.

22. The method of claim 16, further comprising steps of:
Monitoring wound healing after each treatment using a near infrared optic device that provides diagnostic information about the healing progress, and
based on the diagnostic information, deciding whether to increase or decrease the number of treatments, duration of a treatment or another operational parameter of the treatment.

23. The method of claim 16, wherein the wound is an ulcer.

24. The method of claim 16, wherein the device is positioned against the epidermal tissue of the patient proximate to the site of the wound for up to 15 minutes to achieve a therapeutic effect with ultrasonic waves having a frequency of 10-100 kHz.

25. The method of claim 16, wherein the device is positioned against the epidermal tissue of the patient proximate to a site of the wound for up to 15 minutes to achieve a therapeutic effect with ultrasonic waves having a frequency of 20-100 kHz.

26. The method of claim 16, wherein the conductive material bonding the first and second covers to the piezoelectric element is a conductive epoxy and each of the first and second covers comprises a base bonded to the piezoelectric element, an apex spaced apart from the base and an inclined wall connecting the base to the apex, wherein the apex is in a circular configuration.

27. The method of claim 16, wherein an angle of inclination of the inclined wall relative to the base of each of the first and second covers increases when the piezoelectric element expands.

28. The method of claim 16, wherein a distance between the piezoelectric element and the apex of the first cover is 0.01 mm to 5 mm.

29. The method of claim 16, wherein the device has an excitation voltage to ultrasound wave amplitude efficiency of at least 20V:55 kPa/100 mWcm².

30. The method of claim 16, wherein the ultrasound transducer is mounted to a flexible wire, and wherein the electronic driving module is removably attached to ultrasound transducer.

31. The method of claim 16, wherein the device comprises an array of transducers which is removably mounted to the device.

32. The method of claim 16, wherein the device is a flexible patch or bandage that can be worn by a patient and the device weighs 200 grams or less.

33. The method of claim 16, wherein the electrical matching network comprises inductors and/or resistors.

34. The method of claim 16, wherein the device further comprises a housing having an opening, wherein at least one ultrasound transducer is potted within the opening with an epoxy.
